(19) United States
(12) Patent Application Publication
(10) Pub. No.: US 2011/0277903 A1
(43) Pub. Date: Nov. 17, 2011

(54) METHOD AND APPARATUS FOR REDUCING HEAT RELEASE AND SMOKE DENSITY

(75) Inventor: Kirk R. ADAMS, Smyrna, GA (US)

(73) Assignee: GLOBAL ONBOARD PARTNERS, LLC, Atlanta, GA (US)

(21) Appl. No.: 13/084,839

(22) Filed: Apr. 12, 2011

Related U.S. Application Data
(63) Continuation-in-part of application No. PCT/US2009/056207, filed on Sep. 8, 2009.
(60) Provisional application No. 61/095,148, filed on Sep. 8, 2008.

(51) Int. Cl. B32B 37/02; B32B 3/18; G01N 9/00; B32B 18/00; G01N 33/00; B32B 3/14; B32B 15/00

(52) U.S. Cl. 156/60; 156/64; 428/195.1; 428/203; 428/209; 428/210; 73/865.8; 73/30.01

(57) ABSTRACT

A system and method for combining specific fire barrier materials along with graphic materials and interior aircraft material/parts to lower the heat release and smoke density when tested using the FAA regulated OSU Heat Release and Smoke Density Tests and/or any other test as required by any countries government in order to install graphics onto an aircraft material/part.

### CROSS-REFERENCE TO RELATED APPLICATIONS

This application is a continuation-in-part of International PCT Patent Application No. PCT/US2009/056207 filed Sep. 8, 2009, which claims the benefit of U.S. Provisional Patent Application Ser. No. 61/095,148 filed Sep. 8, 2008; which are incorporated herein by reference in their entireties for all purposes.

### TECHNICAL FIELD

The present invention relates generally to the fields of fire-resistant materials and display systems, and more particularly to a fire-resistant material system and a related method for reducing heat release and smoke density, and to display systems and method incorporating such a material system.

### BACKGROUND

In the United States, the OSU Heat Release Test and apparatus is commonly used to measure the heat release rate (HRR) of materials and products in forced flaming combustion. Similar tests and apparatus may be used by other countries and/or governmental entities, at present or in the future. The OSU test measures oxygen consumption and sensible enthalpy (temperature rise) of the apparatus in addition to the usual sensible enthalpy of smoke density and/or the exhaust gases during the test. The test is used in the aircraft field to expose aircraft interior cabin materials (parts) to an incident radiant heat flux of approximately 35 kW/m2 in order to comply with FAR 25.853 [a-1] requirements. In order to pass this test under currently recognized parameters, interior cabin materials (parts) must not provide a heat release measurement which exceeds 65 kW/m² at a peak or 65 kW/m² over a two minute period (<65/65) to pass. An apparatus or test similar to the OSU Heat Release Test and apparatus may be used by the FAA or other countries' regulatory or governmental entities to test compliance with other present or future applicable regulations or standards.

Many interior aircraft parts burn at or near the 65/65 HRR measurement on their own. Thus, if even a single layer of most any type of previously known graphic display material is added or adhered to the aircraft part, the measurement will typically exceed the OSU 65/65 measurement, often times producing heat release in excess of 80 kW/m2 (+/−) at a peak and 80 kW/m2 (+/−) over a 2 minute period or 80/80 (+/−), resulting in a failed test and eliminating the possibility for an airline or media company to be able to install the graphic display material onto that aircraft interior material/part. When previously known graphic display material is applied to airline parts and undergoes these tests with their stringent levels and criteria, the heat release and smoke density typically dramatically increase, resulting in failure of the test, and limiting the potential areas where graphic display materials can be applied on an aircraft.

The Smoke Density Test required by the FAA results in a failure for conditions that produce Max Ds (maximum specific optical density) at four minutes>200. This means that smoke density from any aircraft interior material/part cannot exceed 200 over a four minute period. When previously known graphic display materials are placed on top of, or adhered to, these aircraft materials/parts and subjected to the Smoke Density test, they commonly exceed the allowable levels of smoke density, thus further inhibiting the application of graphic display materials inside the cabin on airline materials/parts. Similar tests and ways of measuring smoke density may be used by the FAA or other countries' governmental agencies now or in the future to test compliance with applicable regulations or standards.

### SUMMARY

Airlines are constantly seeking ways to increase revenue, especially during times when fuel costs and increased competition make it increasingly more difficult to remain financially viable. Many airlines are turning to non-transportation related revenue opportunities to offset these costs. Presently some airlines use advertising media inside the airplane cabin as a means of increasing non-transportation related revenue. Accomplishing this can provide an airline with millions of dollars in increased revenue, thus leading to greater job security for employees and greater overall financial strength and viability for the airline. Known forms of advertising inside an airliner cabin environment are typically limited to areas such as the back or front of the tray tables, which only require passage of the FAA's Vertical Burn Test, or similar tests which can be passed by displays applied using standard graphic display materials. It is also noted that use of the new invention when used on the tray tables greatly reduces the possibility of smoke or fire in the cabin due to its unique properties, enhancing its ability to make the cabin safer over any previous art specific to the tray table.

The challenge comes when an airline or media company desires to place display materials such as graphics or advertisements in areas inside the cabin such as the overhead bin doors, sidewall panels, bulkhead/windscreens, and other areas on the interior of the aircraft which require testing beyond the Vertical Burn Test standards and to meet the strict standards of the FAA and other governmental entities. These additional tests include the Ohio State University (OSU) Heat Release and Smoke Density Tests, or other tests set forth by other countries' governmental agencies. Such tests are typically required prior to placing display materials on aircraft material/parts where the size of the graphic material exceeds 6″×6″ for heat release and 3″×3″ for smoke density. Other size requirements may be less than or greater than 6″×6″ for heat release and 3″×3″ for smoke density testing, as determined by the applicable country's government agency or standards, compliance with which is within the scope of this invention. To applicant's knowledge, no previously known materials have permitted the application of display materials to be directly adhered to aircraft cabin parts in compliance with these tests and standards.

The present invention provides a material system that allows an airline or media company or other entity to comply with the required FAA OSU tests and/or other tests required by any government regulation or standard, to enable the application of various forms of displays on aircraft material/parts. Example forms of the present invention allow the application of display materials directly onto aircraft cabin parts in compliance with the OSU Heat Release and Smoke Density Tests and have received the first ever passing test results from the FAA for such materials, allowing such display materials to be placed on commercial registered airline aircraft as a direct result of the present invention. With the creation of this method and apparatus for reducing heat release and smoke density allowing for this invention to be adhered directly to an aircraft cabin part, a Supplemental Type Certificate or other government documentation is accepted by other governmental entities around the globe according to bi-lateral agreements between countries. This makes the invention that much more valuable as it is accepted and can be used to benefit a greater number of airlines around the globe. As such, the present invention is expected to provide the same or similar benefits when applied to similar tests by other countries' governmental agencies requiring a reduction in the amount of heat release and/or smoke density tests in order to install displays on aircraft material/parts.

By incorporating this invention, airlines, media companies or any other entity performing these tests will be able to see the necessary heat release and smoke density reductions, allowing them to pass each test and further enabling airlines to generate additional non-transportation related revenue, which in turn will provide greater financial stability, providing greater job security and viability of the airline.

According to example forms of the invention, a fire barrier material or composition is sandwiched between the display and the aircraft part. The fire barrier material can also be placed directly onto the aircraft materials/parts in a more permanent fashion. In the event that the barrier is placed directly onto the aircraft materials/parts, it only needs to be changed out in the event it becomes torn or damaged. The barrier dramatically reduces the ability for fire to penetrate the barrier and reach the aircraft materials/parts, as well as lowers the overall thermal conductivity. As a direct result of this invention, when fire is unable to fully reach the aircraft material/parts, the amount of heat and/or smoke density released is dramatically inhibited and reduced, allowing the graphic material adhered to the aircraft material/parts to pass the required FAA and any other tests required by any country's government to accomplish the same goal of installing displays on aircraft material/parts. When the aircraft material/part does not catch on fire, the smoke density is dramatically reduced, allowing the airline/media company to pass the stringent Smoke Density Test.

Example embodiments of the invention dramatically inhibit the fire from transferring from the graphic material into the interior airline material/part. Example embodiments of the invention lower the heat release when the graphic material is adhered to the barrier material which is adhered to or placed on the interior airline material/part. Example embodiments of the invention lower the smoke density produced when the graphic material is placed in front of the barrier, which is placed or adhered on the airline material/part. Example embodiments of the invention do not suffer from the problems of the previous deficiencies that existed when trying to pass the FAA or other countries governmental required tests when combining graphic material with interior aircraft material/parts.

In one aspect, the invention relates to a graphic display material having a fire barrier material with a display information material applied onto it. The fire barrier material is selected from one or more of: a ceramic-based material, a ceramic based paint, an insulating thermal barrier coating, an aluminum material, a polymeric flame resistant material, an aramid, a hybrid aramid/inorganic fiber based material, and a melamine-based fiber and/or combinations thereof.

In another aspect, the invention relates to an aircraft component having a fire barrier material for receiving display information material applied onto it. The fire barrier material is selected from one or more of: a ceramic-based material, a ceramic based paint, an insulating thermal barrier coating, an aluminum material, a polymeric flame resistant material, an aramid, a hybrid aramid/inorganic fiber based material, and/or a melamine-based fiber.

In still another aspect, the invention relates to a method of displaying information display material on a surface having several steps. A fire barrier material is applied to a surface with adhesive. A display material is applied to the fire barrier material with adhesive. And a transparent outer layer is applied to the display material with adhesive.

In still another aspect, the invention relates to a method of reducing thermal conductivity, heat release and/or smoke density. A fire barrier product is applied behind a graphic material. The fire barrier product is submitted to, and passes, an OSU Heat Release Test.

In another aspect, the invention relates to a surface covering material including a plurality of layers. The layers include at least two materials selected from a fire barrier material, a graphic display material, and a transparent cover material.

In another aspect, the invention relates to a graphic display laminate for application to airline components. The laminate includes an inner fire-barrier layer of at least one material selected from a ceramic-based material, a ceramic based paint, an insulating thermal barrier coating, an aluminum material, a polymeric flame resistant material, an aramid, a hybrid aramid/inorganic fiber based material, a melamine-based fiber, and/or combinations thereof. The laminate also includes a transparent, translucent or otherwise light-transmissive outer protective layer. The laminate also includes a graphic display layer between the inner fire-barrier layer and the light-transmissive outer protective layer.

These and other aspects, features and advantages of the invention will be understood with reference to the detailed description herein, and will be realized by means of the various elements and combinations particularly pointed out in the appended claims. It is to be understood that both the foregoing general description and the following detailed description of the invention are exemplary and explanatory of preferred embodiments of the invention, and are not restrictive of the invention, as claimed.

### DESCRIPTION OF THE FIGURES

FIG. 1 shows displays incorporating fire barrier material in an airplane cabin, according to example forms of the present invention.

FIG. 2 shows an exploded side cross-sectional view of a fire barrier display system for application to the surface of an airline storage compartment, according to an example form of the invention.

FIG. 3 shows an enlarged cross-sectional view of a portion of the fire barrier system shown in FIG. 2.

### DETAILED DESCRIPTION OF EXAMPLE EMBODIMENTS

The present invention may be understood more readily by reference to the following detailed description of the invention. It is to be understood that this invention is not limited to the specific devices, methods, conditions or parameters described herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

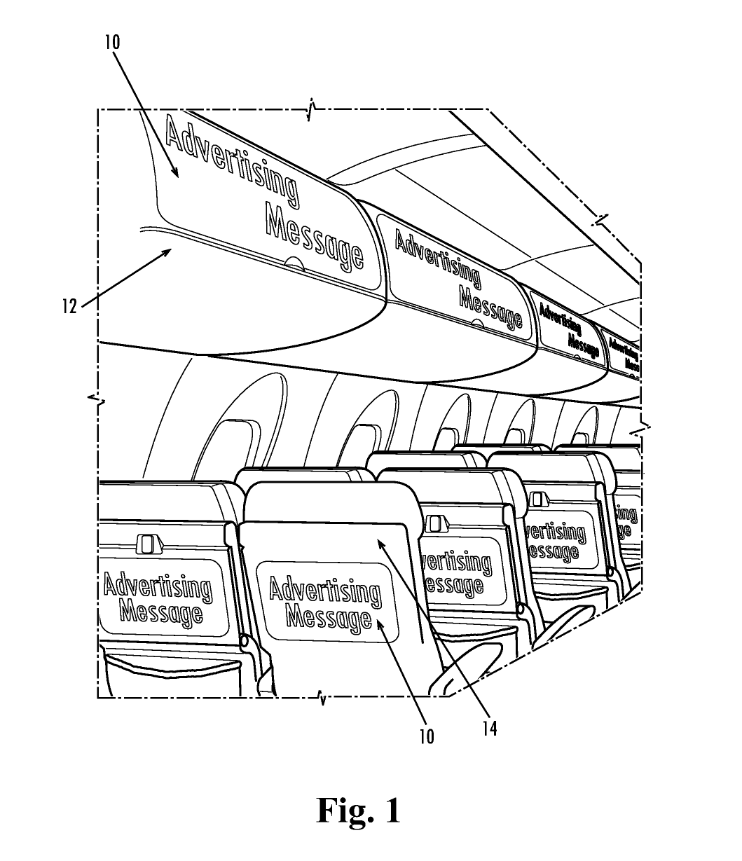

FIG. 1 shows an example embodiment of a fire barrier display material 10 having displayed material such as advertising or informational text and/or graphics in use within an airplane cabin. The fire barrier display material 10 can be affixed to the overhead storage compartment surface 12, the back of a seat or fold-up table 14, bulkhead walls, cabin ceilings, sidewall panels, and/or other cabin surfaces. The fire barrier display material 10 acts as a fire barrier or inhibitor, assists in lowering thermal conductivity and heat release, and/or lowers smoke density. The fire barrier display material 10 includes one or more layers of fire barrier compound or other fire-resistant product underlying a layer of graphic display material or other form of display surface, creating a graphic sandwich or layered laminate system, wherein the graphic is placed on top of and adhered or otherwise affixed to the fire barrier material/compound used to reduce thermal conductivity, heat release and smoke density. The fire barrier compound or material is adhered or otherwise attached to the aircraft part 12. The graphic display material can be permanently affixed to the fire barrier compound or material, such that when the graphic display material is changed out, the fire barrier compound or material is changed out as well. Alternatively, the attachment between the graphic display material and the fire barrier compound or material is releasable, whereby the graphic display material can be removed and replaced, with the fire barrier compound or material remaining in place.

The fire barrier compound or material is also used as a base template where it is applied directly to the aircraft part and the graphic/advertisement material is applied on top of the fire barrier compound or material. When the graphic is removed or changed out, the fire barrier compound or material remains adhered to/attached to the aircraft part; or alternatively, is replaced or repaired if damaged during the removal process.

The fire barrier compound or material is placed behind any of a variety of types or styles of advertising graphic display material which can be printed on directly. These display materials include, but are not limited to vinyl, polyester synthetic, and/or any other manner of product upon which graphics can be printed. The material upon which the graphic is printed may be covered with a fire retardant and/or graffiti resistant product, resin, layer or coating such as Lexan, Tedlar, polycarbonate, glass, urethane, acrylic or any other desirable and appropriate product, resin, layer, or coating.

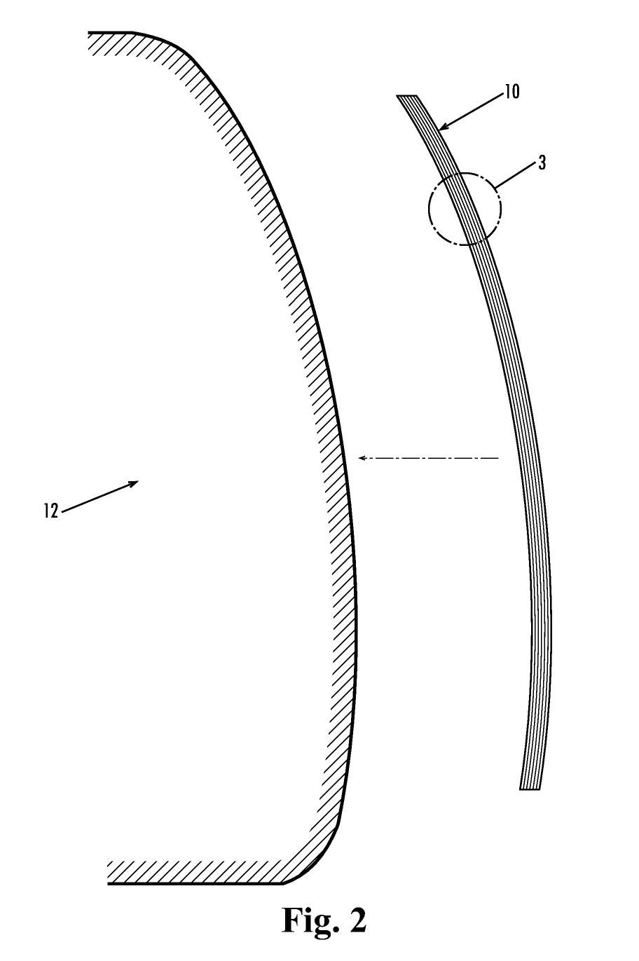

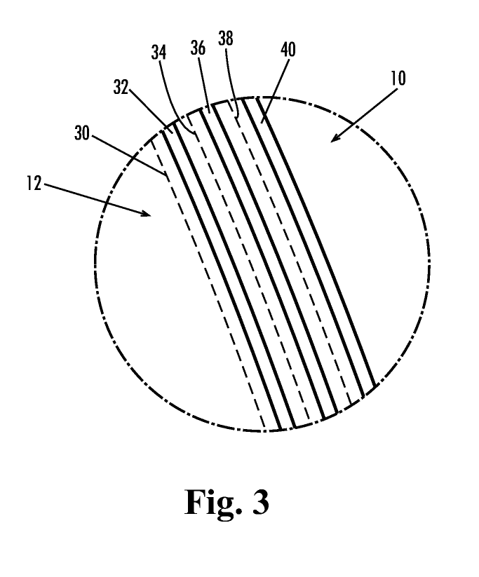

FIG. 2 shows an exploded cross sectional view of an example form of a fire barrier display material 10 according to an embodiment of the present invention, separated from an overhead compartment storage surface 12. As shown, the example embodiment of the fire barrier display material 10 has six layers. FIG. 3 shows an enlarged cross sectional view of the layers of the fire barrier display material 10 as shown in FIG. 2. As shown, the example embodiment has six layers.

An outer layer 40 of transparent material acts as a cover. The outer layer 40 can comprise, for example, Tedlar, Lexan, or other substantially transparent or translucent material or coating, and preferably is fire-resistant and graffiti and tamper-resistant. The outer layer 40 is affixed to a middle layer 36 of display material on which graphics can be printed or otherwise applied. The outer layer 40 is affixed to the middle layer 36 with an adhesive or bonding layer 38 to form a multi-layer laminate structure. The light and/or color transmissive nature of the outer layer 40 is such that printed material on the middle layer 36 is at least partially visible through the outer layer 40, and optionally is clearly and entirely visible therethrough.

The middle layer 36 can comprise, for example, a vinyl, polyester, or other material upon which graphics, text or other display material are or can be printed or otherwise applied. Optionally, the middle layer is a paint, dye, pigment, stain, ink, print-media, foil, film, thermographic print media or other graphic display media applied directly onto the outer layer and/or the inner layer in a self-adhesive fashion, without the need for application of a separate adhesive compound therebetween. The middle layer 36 is affixed to an inner layer 32.

The inner layer 32 is a fire barrier compound or material forming a firewall or fire-resistant barrier, for example comprising a ceramic-based fire barrier, an aluminum-based fire barrier, a polymer-based fire barrier, an aramid-based fire barrier, a hybrid aramid/inorganic fire barrier product, a melamine-based fire barrier, or other form of fire resistant material. The middle layer 36 is affixed to the inner layer 32 with an adhesive layer 34. The inner layer 32 is affixed to the surface 12 with an adhesive layer 30. The fire barrier product 10 inhibits and blocks a fire when affixed to the surface 12.

In alternate embodiments of the invention, the fire barrier display material 10 may optionally comprise any two or more layers selected from the inner layer 32, the middle layer 36 and/or the outer layer 40, each layer comprising at least one material selected from the example materials specified above for the respective layers. For example, embodiments comprising an inner layer material in combination with a middle layer material, a middle layer material in combination with an outer layer material, and/or an inner layer material in combination with an outer layer material, are contemplated to be within the scope of the invention, depending on the desired application and the particular selection of materials.

Placing the fire barrier material/product/compounds either in a sandwiched manner between the graphics materials and the airplane materials/parts or placed as a template barrier directly on the aircraft materials/parts may include any one or more combinations of the following materials, or any similar fire barrier materials that assist in lowering both heat release and smoke density when tested using the FAA OSU Heat Release and Smoke Density tests or any other test required by any countries governmental entity in order to allow an airline or media company to install graphics onto aircraft material/parts: ceramic-based felts, fibers, papers, and materials, which include alumina-silicate based products, and products within this same chemical composition family, are primarily made of Al₂O₃, SiO₂, and/or B₂O₃, but can include any other ceramic based chemical composition which satisfactorily creates a fire barrier and assists in lowering thermal conductivity from the graphic into the airplane part as well as incorporating any additional benefits these products may provide.

The fire barrier layer thickness can be any range of thickness that maintains the fire barrier and continues to provide the necessary reductions in thermal conductivity required to pass the OSU Heat Release and Smoke Density Tests, or any other test any countries governmental entity may require to install graphics onto aircraft material/parts. Any layer of density is within the scope of the invention as long as it continues to perform as necessary under the fire and smoke conditions as outlined in the OSU Heat Release and Smoke Density tests and/or any other tests required by any countries government entity to accomplish the goal of this invention. The particular parameters of the barrier layer will be determined based on the selected materials of construction and the intended application or manner of use.

These barriers can typically resist continuous temperatures exceeding 1800 degrees Fahrenheit, as is required under the OSU Heat Release guidelines, or other countries governmental tests required to place graphics onto the interior aircraft materials/parts. One of the most significant attributes of Ceramic-based compounds is that they are typically not burned or melted in the event of a fuel fire.

In alternative embodiments, ceramic based paint and insulating thermal barrier coatings provide a strong heat reflecting, insulating, thermal barrier that can be sprayed directly onto the aircraft material/parts or applied directly onto a substrate that can then be applied as part of the invention in sandwich form, placed behind the graphic materials to apply directly to the aircraft materials/parts or as a base template, where it is applied directly to/onto the aircraft material/part in a more permanent fashion and the graphics are applied and removed from off the template. The ceramic based paint or coating can be in the form of a paint additive that when combined with paint creates the necessary radiant heat reflecting, insulating, thermal barrier coating or it can come as a liquid form to be sprayed or applied or acquired and applied to the aircraft material/parts or in the sandwich form as a pre-sprayed/formed solid material that presents the same heat reflecting, insulating, thermal barrier properties as well as incorporating any additional benefits these products may provide. It has been noted that the non-tumescent ceramic based paint, coatings or materials may provide better results, but intumescent and non-intumescent paints, coatings, and materials are within the scope of the invention.

In still further embodiments of the invention, aluminum foils and/or tapes adhere directly to any surface and provide fire resistant properties that assist with lowering thermal conductivity and smoke density. They can be cut in standard strips and provided on rolls, or provided as a specialty cut up the desired width and length.

Additional embodiments of the invention comprise fire barrier layers selected from one or more polymer-based and/or polymeric flame-resistant materials, including without limitation, oxidized polyacrylonitrile-based (PAN) felt/product, aramid-based felt/product, melamine-based fabric, or hybrid aramid/inorganic fiber/felt/product. Such materials act as a sufficient barrier against fire and reduce thermal conductivity in the event of a fire in the aircraft cabin as well as incorporating the many additional benefits these materials and products may provide. PAN-based fiber can be blended with other fibers to produce a wide variety of felted, paper, or other product materials/compounds that provide excellent burn-through protection properties. These materials can be produced using numerous methods including ammoxidation or propylene.

Aramid is an abbreviation for aromatic polyamide. The chemical composition of a commercial aramid is poly-para-phenylene-terephthalamide. It may also be known as para-aramid. Aramid fiber/product is a man-made organic polymer produced by spinning a solid fiber from a liquid chemical blend. The bright golden yellow filaments produced have high strength and low density giving very high specific strength. Aramid materials do not ignite, melt, or drip, giving exceptionally beneficial characteristics when used in the cabin environment behind a graphic. Aramid materials are extremely flame-resistant and maintain extremely high tensile strength as well as incorporating the many additional benefits these materials and products may provide.

Hybrid aramid/inorganic fiber/product is another type of fire barrier material resulting from the combination between aramid material and ceramic oxides is the hybrid aramid/inorganic fiber/product. This material is usually formulated with the Nitrile Rubber (NBR) binder and exhibits a high strength, low thermal conductivity, and excellent fire resistance and incorporates the many additional benefits these fibers/products/materials may provide.

Melamine-based fiber/product can also be used to enhance the performance of fire blocking materials. It exhibits excellent heat insulation properties and provides low thermal conductivity. The distinct diameter distribution of these fibers can trap insulating air, further increasing the heat blocking characteristics of fire blocking fabrics, papers and other compounds, which can greatly assist in lowering heat transfer during the OSU Heat Release test and any other test required by any countries government entity in order to install graphic materials onto any interior aircraft material/part.

Many variations of the invention will be understood by those skilled in the art. Example variations include placing the barrier in different locations within the sandwich, for example as a middle or outer layer. Other variations call for additional ways in which the product can be applied to the aircraft materials/parts. Even further variations can include multiple combinations of the barriers or ways in which they are applied or included in the invention. Other variations include fire barrier, and other materials not listed within this document, that when placed within the layered formation of this invention within a sandwich or laminate structure as indicated for example between the graphic materials and the interior aircraft material/parts, provides the protection against fire as well as a reduction in heat release and smoke density that assists the airline/media company when passing the OSU Heat Release and other countries governmental tests. All such variations are intended to be within the scope and spirit of the invention.

Although some embodiments are shown to include certain features, the applicant(s) specifically contemplate that any feature disclosed herein may be used together or in combination with any other feature on any embodiment of the invention. It is also contemplated that any feature may be specifically excluded from any embodiment of an invention.

Further, a person skilled in the art will realize that other embodiments of the invention, and modifications, additions and deletions thereto, are possible and that the details of the invention can be modified in a number of respects, all without departing from the inventive concepts set forth in the claims. Thus, the above description is to be regarded as illustrative in nature and not restrictive.

What is claimed is:

1. A method of displaying informational material on a surface, comprising:
applying a fire barrier material to the surface;
wherein said fire barrier material is selected from a ceramic-based material, a ceramic based paint, an insulating thermal barrier coating, an aluminum material, a polymeric flame resistant material, an aramid, a hybrid aramid/inorganic fiber based material, a melamine-based fiber, and combinations thereof; and
applying a display material to the fire barrier material.

2. The method of claim 1, wherein the surface is an aircraft component.

3. The method of claim 1, wherein the fire barrier material comprises a ceramic-based material.

4. The method of claim 1, wherein the fire barrier material comprises a ceramic based paint.

5. The method of claim 1, wherein the fire barrier material comprises an insulating thermal barrier coating.

6. The method of claim 1, wherein the fire barrier material comprises aluminum.

7. The method of claim 1, wherein the fire barrier material comprises a polymeric flame resistant material.

8. The method of claim 1, wherein the fire barrier material comprises an aramid.

9. The method of claim 1, wherein the fire barrier material comprises a hybrid aramid/inorganic fiber.

10. The method of claim 1, wherein the fire barrier material comprises a melamine-based product.

11. The method of claim 1, wherein the fire barrier material is first adhered to the surface.

12. The method of claim 1, wherein the fire barrier material is first adhered to the display material.

13. The method of claim 1, further comprising carrying out a test on a representative sample of the surface bearing the fire barrier material and the display material, wherein the properties tested are selected from one or more of: heat release, smoke density and combinations thereof.

14. The method of claim 1, further comprising applying a transparent outer layer onto the display material.

15. The method of claim 14, further comprising:
applying an adhesive between the surface and the fire barrier material;
applying an adhesive between the fire barrier material and the display material; and
applying an adhesive between the display material and the transparent outer layer.

16. The method of claim 1, wherein the fire barrier material is permanently affixed to the surface and the display material is removable from the fire barrier material.

17. A display comprising a fire barrier material having a display material applied thereon, wherein the fire barrier material is selected from a ceramic-based material, a ceramic based paint, an insulating thermal barrier coating, an aluminum material, a polymeric flame resistant material, an aramid, a hybrid aramid/inorganic fiber based material, a melamine-based fiber, and combinations thereof.

18. An aircraft component comprising a fire barrier material for receiving a display material applied thereon; wherein the fire barrier material is selected from a ceramic-based material, a ceramic based paint, an insulating thermal barrier coating, an aluminum material, a polymeric flame resistant material, an aramid, a hybrid aramid/inorganic fiber based material, a melamine-based fiber, and combinations thereof.

19. A method of verifying compliance with a standard, said standard specifying at least one characteristic of an object when burned, said method comprising:
applying a display to an object;
burning at least a portion of the object having the display applied thereon; and
carrying out a test regimen specified by the standard on the object.

20. The method of claim 19, wherein the standard is a heat release test.

21. The method of claim 20, wherein the standard is an OSU Heat Release Test.

22. The method of claim 19, wherein the standard is a smoke density test.

23. The method of claim 19, wherein the object is an aircraft component.

24. A method of displaying informational material on a surface, comprising:
applying a fire barrier material to a surface with adhesive;
applying a display material to the fire barrier material with adhesive; and
applying a transparent outer layer to the display material with adhesive.

25. A graphic display material for application to a substrate, said graphic display material comprising:
a fire-barrier layer; and
a printable display material overlying the fire-barrier layer.

26. The graphic display material of claim 25, further comprising a transparent cover layer overlying the printable display material.

27. The graphic display material of claim 25, further comprising an adhesive applied between the fire-barrier layer and the printable display material.

28. The graphic display material of claim 25, wherein the graphic display material is capable of passing a regulatory heat-release test.

29. A surface covering material comprising a plurality of layers, said layers comprising at least two materials selected from a fire barrier material, a graphic display material, and a transparent cover material.

30. The surface covering material of claim 29, further comprising an adhesive layer between the at least two layers.

31. A graphic display laminate for application to airline components, said laminate comprising:
an inner fire-barrier layer comprising at least one material selected from a ceramic-based material, a ceramic based paint, an insulating thermal barrier coating, an aluminum material, a polymeric flame resistant material, an aramid, a hybrid aramid/inorganic fiber based material, a melamine-based fiber, and combinations thereof;
a light-transmissive outer protective layer; and
a graphic display layer between the inner fire-barrier layer and the light-transmissive outer protective layer.

32. The graphic display laminate of claim 31, wherein the inner fire-barrier layer, the light-transmissive outer protective layer and the graphic display layer are adhered to one another to form a flexible sheet.

33. The graphic display laminate of claim 32, further comprising an adhesive backing for attaching the flexible sheet to an aircraft cabin component.

34. The graphic display laminate of claim 31, wherein said laminate passes the OSU Heat Release Test.

35. The graphic display laminate of claim 31, wherein said laminate passes the OSU Smoke Density Test.
